(19) United States
(12) Patent Application Publication
(10) Pub. No.: US 2005/0216208 A1
(43) Pub. Date: Sep. 29, 2005

(54) Diagnostic decision support system and method of diagnostic decision support

(76) Inventors: Akira Saito, Kokubunji (JP); Satoshi Mitsuyama, Tokyo (JP); Hideyuki Ban, Kodaira (JP)

Correspondence Address: Mattingly, Stanger & Malur. P.C., Suite 370, 1800 Diagonal Road, Alexandria, VA, 22314, US

(21) Appl. No.: 10/901,215

(22) Filed: Jul. 29, 2004

(30) Foreign Application Priority Data
Mar. 26, 2004 (JP) 2004-091104

(51) Int. Cl. G06F 17/60; C12Q 1/68; G06F 19/00; G01N 33/48; G01N 33/50

(52) U.S. Cl. 702/20; 705/3

(57) ABSTRACT

There is provided a system performing high-accuracy diagnostic decision support in consideration of the influence of a haplotype block and a genetic structure.

Haplotype block inference means 13 infers the positions of haplotype blocks, and analyzes each of the haplotype blocks to infer a haplotype pattern of individuals with high accuracy. Genetic structure inference means 15 performs clustering the individuals on the basis of the haplotype pattern to divide a population into some subpopulations, and removes the influence of a genetic structure existing in the population. A genetic structure information database 16 and a clinical information database 11 are used to analyze association of clinical information with genetic information for providing high-accuracy diagnostic decision support knowledge. On the basis of the diagnostic decision support knowledge obtained by analyzing the association of clinical information with genetic information, risk calculation means 19 calculates a risk that a predetermined individual is affected by disease.

### CLAIM OF PRIORITY

The present application claims priority from Japanese application JP 2004-091104 filed on Mar. 26, 2004, the content of which is hereby incorporated by reference into this application.

### FIELD OF THE INVENTION

The present invention relates to a diagnostic decision support system and a method of diagnostic decision support which can analyze association of clinical information with genetic information and sample and show clinically useful information.

### BACKGROUND OF THE INVENTION

The human genome project has almost completed sequence decision to move into the age of post-sequencing. From now on, the effective utilization of an enormous amount of stacked genetic information in medical science is expected. The advancement of clarification of association of genes with disease makes it possible to predict disease-appearing risk on the basis of the genotype of an individual, which enables prevention, early discovery and treatment of the disease according to the genetic predisposition of the individual. To realize these, it is necessary to analyze association of clinical information with genetic information.

As one of strong methods of analyzing association of clinical information with genetic information, there is a method of statistical genetics. The method of statistical genetics is a method of using genetic information and the presence or absence of disease of an individual as data to search for disease-associated genes employing statistics. It may also find disease-associated genes whose mechanism is unknown, which is increasingly important. The method of statistical genetics is a technique for searching for a genetic region associated with a specific trait using a linkage between a plurality of loci (positions of genes on a chromosome). The trait refers to various formative characteristics observed at individual level and is the presence or absence of affected disease, height and the color of eyes or hair. The linkage is an exception to the Mendel's law of independence: “Two different traits are isolated and independent to be inherited.

When loci defining two traits exist on a chromosome to be close to each other, the genes are not isolated and independent and are inherited from parent to child in a linked state. This state refers to a linkage between two loci. In meiosis, partial exchange may occur between a pair of chromosomes passed from parents and a combination of genes passed to their child may be different from that derived from the parents. This phenomenon is called recombination.

The probability that recombination occurs between two loci in one meiosis is called a recombination fraction. As the two loci are closer to each other, the recombination fraction is small. That is, the possibility of their linkage is high. The method of statistical genetics examines, on the basis of recombination information, the presence or absence of a linkage between polymorphism (such as single nucleotide polymorphism and microsatellite) and disease-associated genes over a chromosome to close in on disease-associated loci.

Some methods of statistical genetics have been reported. As for genetic disease, a number of causal genes have been identified by parametric linkage analysis using data of a large pedigree. In the future study of searching for disease causal genes, searching for causal genes of complex disease appearing by a plurality of genetic effects and environmental effects is considered to be the mainstream. It is initially considered that the causal genes of complex disease can be identified by nonparametric linkage analysis (affected sib-pair analysis) using data of a number of small pedigrees. In general, it is often difficult to directly identify the causal genes of complex disease having low penetrance (disease-appearing probability). In recent years, due to its high power and analyzing facilitation, attention has been given to association analysis comparing allele frequencies of polymorphism noted in a case group and a control group.

In the prior art association analysis, the possibility that a gene truly associated with a trait may be missed or a gene not associated with a target trait may be selected by mistake is relatively high. In general, the former is handled as a false negative problem and the latter is handled as a false positive problem. The reasons why false negative and false positive analyzed results are given are as follows: only a haplotype of single polymorphism or polymorphism in a narrow range is used to analyze association of a gene with a trait; no haplotype blocks are considered when performing analysis using haplotype; and no diversity existing in a target group (hereinafter, called a genetic structure) is considered.

The haplotype refers to a combination of alleles derived from the same parent in a plurality of linked loci. Alleles in a plurality of loci existing on a chromosome to be close to each other are transferred to the next generation in a linked state without being influenced by recombination in heterogenesis. After heterogenesis many times, there is found association of a plurality of loci existing to be close to each other. This state is called linkage disequilibrium. In recent years, for instance, Non-patent Document 1 (Gabriel SB et al.: The Structure of Haplotype Blocks in the Human Genome, Science, Vol. 296, pp. 2225-2229, 2002) has reported that there alternately exist on a genome a part called haplotype block in which linkage disequilibrium is maintained in a relatively strong state and a part called hotspot weakening linkage disequilibrium between loci since recombination occurs at high frequency.

This fact means that if the position of a haplotype block can be correctly inferred, an exact haplotype pattern can be decided only by measuring the genotype of a few loci in the haplotype block. At the same time, this fact means that when performing analysis using a plurality of loci across a hotspot, many false positive results which are not important in genetics are given.

When generally performing association analysis, a target population is often divided into groups according to a noted trait. Most famous is case-control study which samples a number of cases and controls from a certain population, compares frequencies of noted alleles of a case group and a control group, and detects loci of polymorphism having significant difference in allele frequency. The case-control study assumes that the case group is perfectly matched with the control group other than a noted trait.

The assumption is not always established, and is a problem when a genetic structure exists in a target population. When sampling a case group and a control group from genetically different populations, a genetic structure significantly affects the analyzed result. The influence of the genetic structure of a population will be described using a simple example. For instance, when collecting a case group and a control group having drepanocyte in the U.S., the case group is supposed to include many people derived from Africa and the control group is supposed to include many people derived from Europe. When comparing the two populations without considering the influence of a genetic structure, a number of loci inherently different in allele frequency between African and European people are detected as causal loci of drepanocyte. A genetic structure of a population gives many false positive analyzed results. The genetic structure of the population may also give false negative analyzed results as well as false positive analyzed results.

[Non-patent Document 1] Gabriel S B et al.: The Structure of Haplotype Blocks in the Human Genome, Science, Vol. 296, pp. 2225-2229, 2002

### SUMMARY OF THE INVENTION

As described above, when performing association analysis without considering the influence of a haplotype block and a genetic structure existing in a target population, many false negative and false positive analyzed results are given, significantly affecting the analyzed results. Accordingly, an object of the present invention is to provide a system performing high-accuracy diagnostic decision support in consideration of the influence of a haplotype block and a genetic structure.

In a diagnostic decision support system and a method of diagnostic decision support according to the present invention, haplotype block inference means, on the basis of polymorphism information, infers the position of recombination to infer the positions of haplotype blocks, and analyzes each of the haplotype blocks to infer a haplotype pattern of individuals with high accuracy. The inferred haplotype frequency information and haplotype pattern information of the individuals are stored in a haplotype information database. Genetic structure inference means performs clustering the individuals on the basis of the haplotype pattern to divide a population into some subpopulations, and removes the influence of a genetic structure existing in the population to analyze association of clinical information with genetic information with high accuracy. The result obtained by the genetic structure inference means is stored in a genetic structure information database to analyze the association of clinical information with genetic information using the genetic structure information database and a clinical information database for providing high-accuracy diagnostic decision support knowledge. The diagnostic decision support knowledge obtained by analyzing the association of clinical information with genetic information is stored in a decision support knowledge database. Risk calculation means calculates, on the basis of information of the decision support knowledge database, a risk that a predetermined individual is affected by disease.

In a diagnostic decision support system and a method of diagnostic decision support according to the present invention, a haplotype block inference algorism can infer the position of recombination to infer the positions of haplotype blocks, and analyze each of the haplotype blocks to infer a haplotype pattern of individuals with high accuracy. A genetic structure inference algorism can perform clustering individuals on the basis of the haplotype pattern to divide a population into some subpopulations, and remove the influence of a genetic structure existing in the population to analyze association of clinical information with genetic information with high accuracy.

### BRIEF DESCRIPTION OF THE DRAWINGS

FIG. 1 is a diagram showing a configuration example of a diagnostic decision support system of the present invention;

FIG. 2 is a diagram showing an example of a haplotype block inference program 13 inferring haplotype frequency of a population and diplotypes of individuals;

FIG. 3 is a diagram showing a stored data example of basic information necessary for setting a haplotype block;

FIG. 4 is a diagram showing a storing example of haplotype pattern and haplotype frequency information in each haplotype block;

FIG. 5 is a diagram showing a storing example of the haplotype pattern for each individual;

FIG. 6 is a diagram of assistance in explaining an example in which five haplotypes shown in haplotypes 1 to 5 in a certain haplotype block are observed;

FIG. 7 is a diagram showing a genetic structure inference program 15 inferring a membership proportion of an individual;

FIG. 8 is a diagram showing a storing example of haplotype pattern and haplotype frequency information in each subpopulation;

FIG. 9 is a diagram showing a storing example of membership proportion information of each individual to each subpopulation;

FIG. 10 is a diagram showing a description example of a decision support knowledge database 18; and

FIG. 11 is a diagram showing a system example in which an outside medical institution 112 accesses a diagnostic decision support system 111 of the present invention via connection paths 31, 32 and the Internet 30 to receive diagnostic decision support using the diagnostic decision support system 111 of the present invention.

### BEST MODE FOR CARRYING OUT THE INVENTION

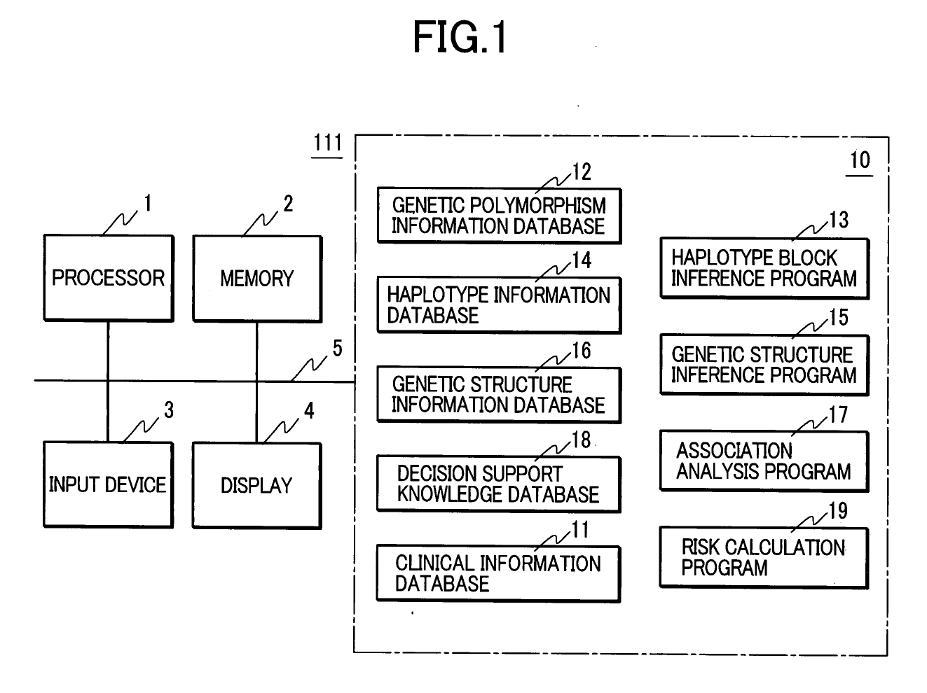

FIG. 1 is a diagram showing a configuration example of a diagnostic decision support system of the present invention. A diagnostic decision support system 111 of the present invention exclusively has an electronic computer such as a personal computer. A system bus 5 is connected to a processor 1, a memory 2, an input device 3, a display 4, and an external memory 10. The external memory 10 incorporates a clinical information database 11 storing clinical information on a plurality of individuals (subjects), a genetic polymorphism information database 12 storing information on polymorphism of the plurality of individuals (subjects), a haplotype information database 14 storing haplotype frequency information of a population and a haplotype pattern of the individuals in each of haplotype blocks obtained by inferring the positions of the haplotype blocks on the basis of information of the genetic polymorphism information database 12 and inferring the haplotype frequency of the population and the haplotype pattern of the individuals in each of the haplotype blocks, a genetic structure information database 16 storing haplotype information of each of divided subpopulations and membership proportion information of each of the individuals to each of the subpopulations obtained by inferring a genetic structure of the population on the basis of information of the haplotype information database 14, performing clustering the individuals on the basis of the haplotype pattern for each of the haplotype blocks, dividing the population into some subpopulations, and inferring the membership proportion of each of the individuals to each of the subpopulations, a decision support knowledge database 18 analyzing association of the haplotype pattern of the individual with a trait for each of the haplotype blocks of the subpopulation on the basis of information of the clinical information database 11 and the genetic structure information database 16 and storing knowledge obtained by association analysis calculating a risk of being affected by disease, a haplotype block inference program 13 leading information of the haplotype information database 14 from information of the genetic polymorphism information database 12, a genetic structure inference program 15 leading information of the genetic structure information database 16 from information of the haplotype information database 14, an association analysis program 17 leading information of the decision support knowledge database 18 from information of the clinical information database 11 and the genetic structure information database 16, and a risk calculation program 19 calculating, on the basis of information of the decision support knowledge database 18, a risk that a predetermined individual is affected by disease. In addition to these, it has a database and a program necessary for serving as a function as an electronic computer.

Data of a population is handled for the databases. Information of the decision support knowledge database 18 is effective to the population. The contents of the databases are further fulfilled by stacking data of persons who have received diagnostic decision.

In the diagnostic decision support system of the present invention, the haplotype block inference program 13, on the basis of polymorphism information, infers the position of recombination to infer the positions of haplotype blocks, and analyzes each of the haplotype blocks to infer a haplotype pattern of individuals with high accuracy. The inferred haplotype frequency information and haplotype pattern information of the individuals are stored in the haplotype information database 14. The genetic structure inference means 15 can perform clustering the individuals on the basis of the haplotype pattern to divide a population into some subpopulations, and removes the influence of a genetic structure existing in the population to analyze association of clinical information with genetic information with high accuracy. The result obtained by the genetic structure inference program 15 is stored in the genetic structure information database 16 to analyze the association of clinical information with genetic information using the genetic structure information database 16 and the clinical information database 11 for providing high-accuracy diagnostic decision support knowledge. The diagnostic decision support knowledge obtained by analyzing the association of clinical information with genetic information is stored in the decision support knowledge database 18. The risk calculation program 19 calculates, on the basis of information of the decision support knowledge database 18, a risk that a predetermined individual is affected by disease.

The clinical information database 11 stores basic data of the name, address, birthday and family structure of an individual, clinical data such as information on the case history, family history, major complaint, findings, examined result, lifestyle, condition process, treatment process and medicine prescription of the individual, and data on an informed consent. The genetic polymorphism information database 12 stores basic information on polymorphism (position, measurement method, polymorphism type (such as SNP or STRP), and allele frequency), the polymorphism measured result of the individual (such as base sequence pattern, homozygote, or heterozygote), identification information of a specimen used in an examination, and specimen management data of a stored state.

The haplotype block inference program 13 will be described. As described previously, linkage disequilibrium is maintained in a relatively strong state in a haplotype block. For instance, as shown in the previously described Non-patent Document 1, the diversity of a haplotype is known to be relatively small in a haplotype block. To infer the position of the haplotype block, it is necessary to define the strength of linkage disequilibrium in a certain region on a genome.

In general, the strength of linkage disequilibrium is often expressed using coefficient of linage disequilibrium D′ between two loci. The present invention, when coefficient of linkage disequilibrium D′ of a plurality of loci in a certain region satisfies the condition of the following equation, defines the region as a haplotype block.
min(|D′|)>0.8

Haplotype frequency of a population and a haplotype pattern of individuals in each inferred haplotype block are inferred. A combination of two haplotypes owned by the individual is called diplotype configuration. Some methods of inferring a diplotype of an individual from genotype data have been proposed. As representative methods, there are a method of using EM algorism as shown in Document: Excoffier L & Slatkin M: Maximum-likelihood estimation of molecular haplotype frequencies in a diploid population, Mol Biol Evol, Vol. 12, pp. 921-927, 1995 and a PHASE method as shown in Document: Stephens M et al.: A new statistical method for haplotype reconstruction from population data, Am J Hum Genet, Vol. 68, pp. 978-989, 2001.

A method of inferring haplotype frequency of a population and diplotypes of individuals using the EM algorism will be described below. A sample having n individuals will be considered now. In the population, a haplotype in a plurality of linked marker loci is considered so that frequency of the population is F=(F₁, F₂, . . . , F_{M}). M is the total number of potential haplotypes. When the marker loci are all SNP loci, the number of loci is L so that M=2^{L}. Genotype observed data in the plurality of linked marker loci of each individual is G=(G₁, G₂, . . . , Gₙ). In many cases, Gᵢ is incomplete data. The number of diplotypes corresponding to Gᵢ is not decided to be one in many cases. In such case, a probability distribution (called a diplotype distribution) on the potential diplotype is defined. For individual i (i=1, 2, . . . , n), the diplotype corresponding to Gᵢ is Dᵢⱼ (j=1, 2, . . . , mi). Here, mi is the number of potential diplotypes to Gᵢ and the maximum value of mi is M.

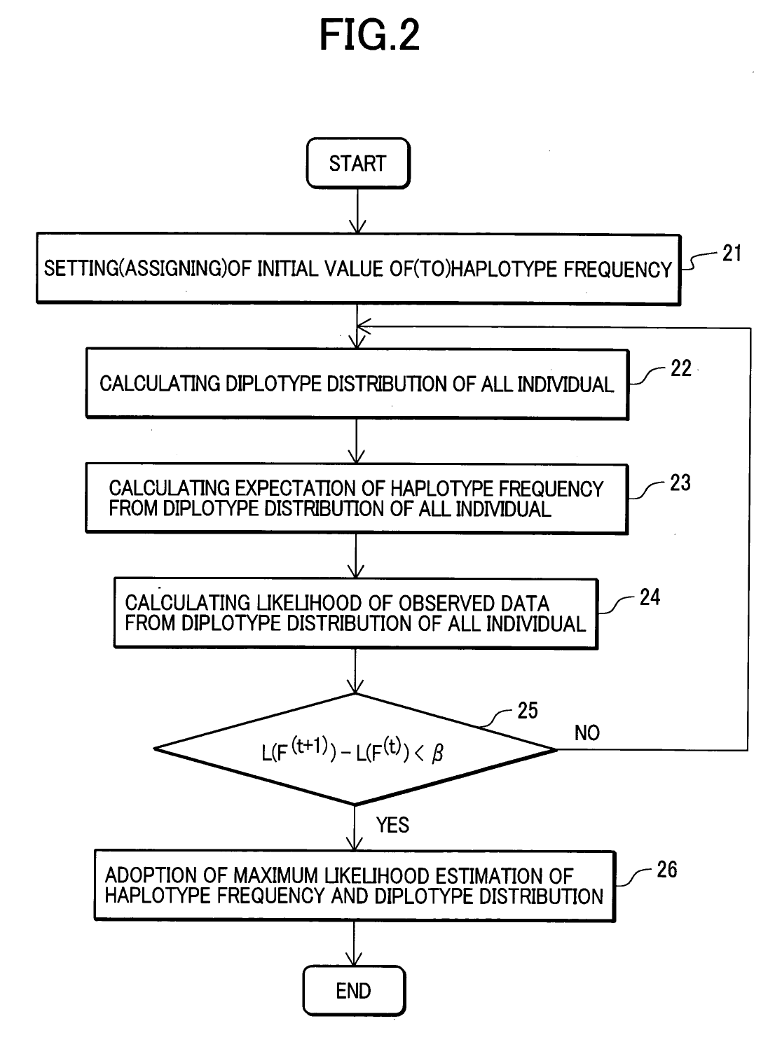

FIG. 2 is a diagram showing an example of the haplotype block inference program 13 inferring haplotype frequency of a population and diplotypes of individuals.

Step 21: Give an initial value F⁽⁰⁾ of haplotype frequency to M potential haplotypes (H₁, H₂, . . . , H_{M}) The total of the haplotype frequency is 1.

For t=0, 1, 2, . . . , calculation for F^{(t)} to F^{(t+1)} is performed by the following steps 22 to 25.

Step 22: Each diplotype Dᵢⱼ has two haplotypes Hₗ, Hₘ where 1≦l≦M and 1≦m≦M. When the haplotype frequency F^{(t)} of a population is given, the probability that Dᵢⱼ is obtained is as shown in Equation (1):

$$Pr ( {D}_{ij} ) = \{ \begin{matrix}{F}_{l}^{{( t )}^{2}} & l = m \\ 2 {F}_{l}^{( t )} {F}_{m}^{( t )} & l \neq m\end{matrix} \qquad ( 1 )$$

Posterior probability Pr(Dᵢⱼ|Gᵢ) that under genotype observed data Gᵢ, the diplotype of individual i is Dᵢⱼ is expressed by Equation (2) by the Bayes' theorem:

$$Pr ( {D}_{ij} \vert {G}_{i} ) = \frac{Pr ( {D}_{ij} ) Pr ( {G}_{i} \vert {D}_{ij} )}{{\sum }_{k = 1}^{{m}_{i}} Pr ( {D}_{ik} ) Pr ( {G}_{i} \vert {D}_{ik} )} = \frac{Pr ( {D}_{ij} )}{{\sum }_{k = 1}^{{m}_{i}} Pr ( {D}_{ik} )} \qquad ( 2 )$$

When this is calculated for all j (j=1, 2, . . . , mi), the diplotype distribution of the individual i is decided. This is applied to all individuals in the sample.

Step 23: When the diplotype distribution of the individual is decided, an expectation of haplotype frequency of the population can be calculated from the diplotype distribution of all individuals in the sample. The expectation of the haplotype frequency of the population is expressed by Equation (3):

$$E [ {F}_{i}^{( t )} ] = \frac{1}{2 n} {\sum }_{j = 1}^{n} {\sum }_{k = 1}^{{m}_{i}} Pr ( {D}_{jk} \vert {G}_{j} ) {N}_{{D}_{jk} i} \qquad ( 3 )$$

where NDⱼₖᵢ is the number of Hᵢ (that is, any one of 0, 1 and 2) included in diplotype Dⱼₖ.

Step 24: The entire likelihood can be expressed by Equation (4) by coupling the likelihood of all diplotypes in each of the individuals and coupling the likelihood of all individuals:

$$L ( {F}^{( t )} ) = Pr ( G \vert {F}^{( t )} ) = {\prod }_{i = 1}^{n} {\sum }_{j = 1}^{{m}_{i}} Pr ( {D}_{ij} ) \qquad ( 4 )$$

Step 25: F is updated as F^{(t+1)}=E[F^{(t)}]. Whether the value of L(F) is converged or not is determined. When satisfying L(F^{(t+1)})−L(F^{(t)})<β, it is converged to advance to step 26. When not satisfying it, the routine is returned to step 22 to repeat until step 25. Here, β is a threshold.

Step 26: E[F]=F^{(EM)} at convergence is maximum likelihood estimation of the haplotype frequency of the population, and Pr(D|G) is the diplotype distribution of the individual under the maximum likelihood estimation of the haplotype frequency of the population.

As described above, the haplotype information database 14 stores haplotype frequency information of a population and a haplotype pattern of individuals for each of haplotype blocks obtained by inferring the positions of the haplotype blocks on the basis of information of the genetic polymorphism information database 12 and inferring the haplotype frequency of the population and the haplotype pattern of the individuals for each of the haplotype blocks, basic information necessary for setting the haplotype blocks, and haplotype pattern and haplotype frequency information in each of the haplotype blocks.

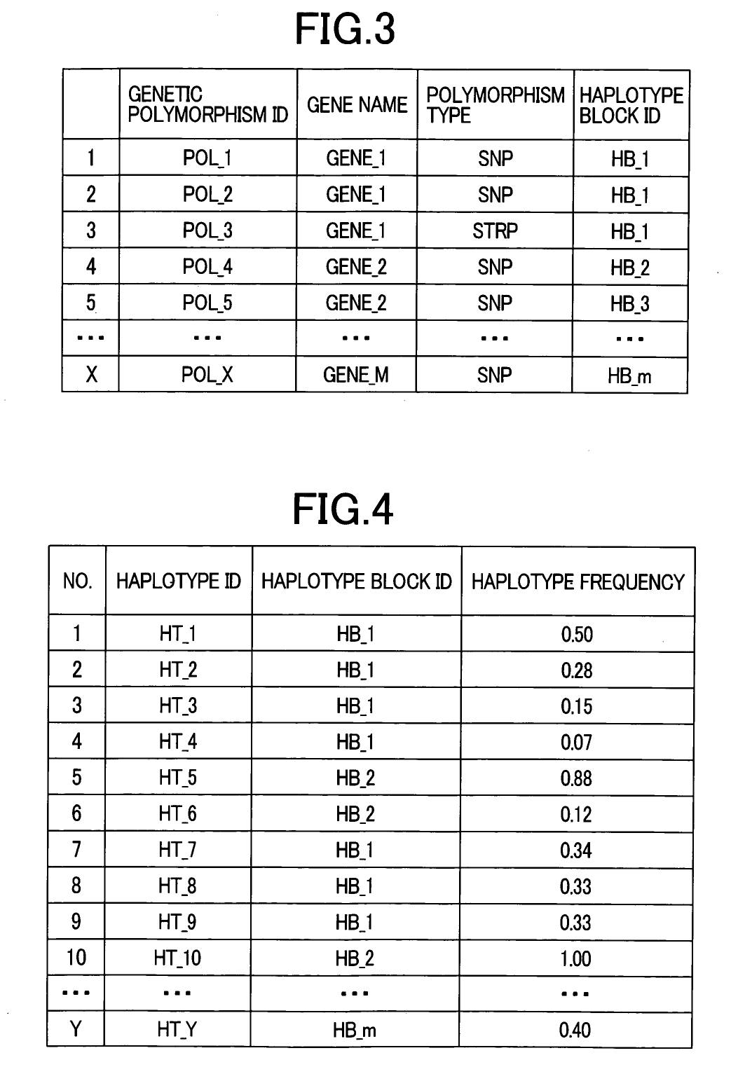

FIG. 3 is a diagram showing a stored data example of basic information necessary for setting a haplotype block. For instance, for gene GENE_1, SNP polymorphism POL_1 and POL_2 and STRP polymorphism POL_3 are registered in a table. POL_1, POL_2 and POL_3 construct haplotype block HB_1. Other than the data shown in FIG. 3, there may be stored the length of the haplotype block, the selection reference of polymorphism constructing a haplotype block (allele frequency and the presence or absence of amino acid variation), coefficient of linkage disequilibrium, and the position of a gene in which polymorphism constructing the haplotype block exists.

FIG. 4 is a diagram showing a storing example of haplotype pattern and haplotype frequency information in each haplotype block. For instance, four haplotypes of HT_1, HT_2, HT_3 and HT_4 exit in haplotype block HB_1. Frequencies of the haplotypes in a population are 0.50, 0.28, 0.15 and 0.07.

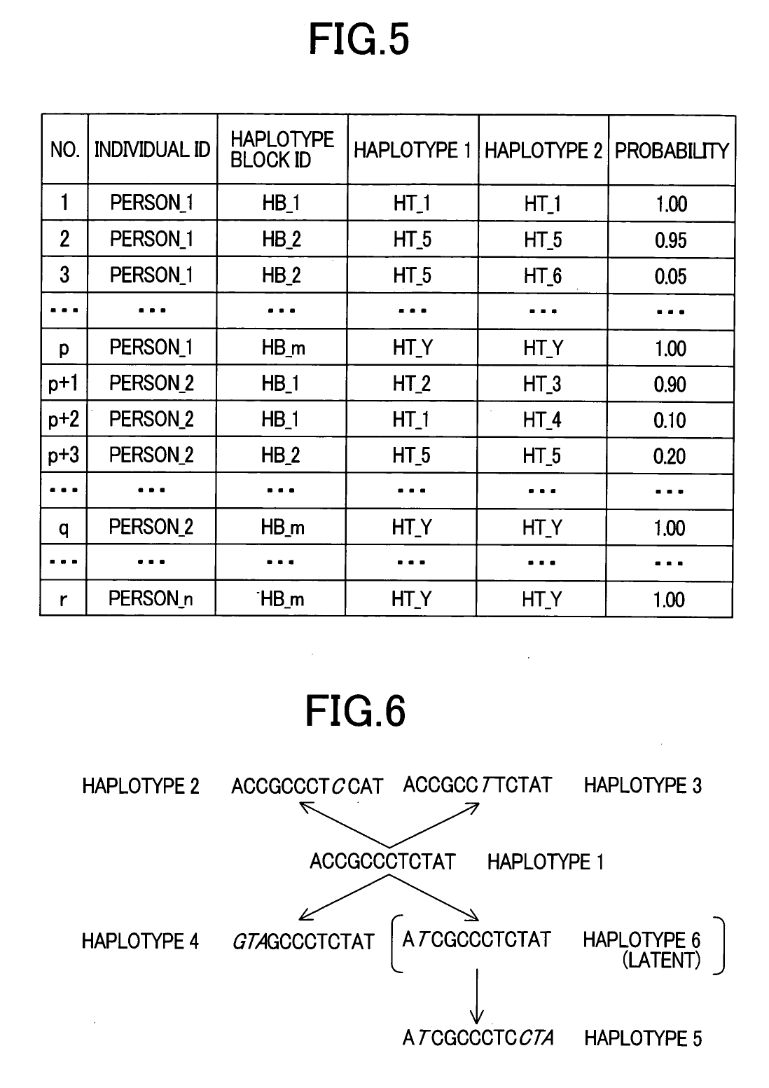

FIG. 5 is a diagram showing a storing example of the haplotype pattern for each individual. For instance, individual PERSON_1 has two haplotypes HT_1 for haplotype block HB_1 (or has a diplotype having two haplotypes HT_1), and the probability of having the diplotype is 1.00. In the same manner, individual PERSON_1 has a diplotype (a probability of 0.95) having two haplotypes HT_5 or a diplotype (a probability of 0.05) having haplotypes HT_5 and HT_6 for haplotype block HB_2. It has a diplotype (a probability of 1.00) having two haplotypes HT_Y for haplotype block HB_m.

The genetic structure inference program 15 will be described. In the present invention, to infer a genetic structure of a population, clustering individuals on the basis of a haplotype pattern is performed to divide the population into some subpopulations. In the present invention, new distance decided by the likelihood of mutation and recombination between haplotypes is defined to use the distance for performing clustering individuals. A clustering method of the present invention will be described below.

FIG. 6 is a diagram of assistance in explaining an example in which five haplotypes shown in haplotypes 1 to 5 in a certain haplotype block are observed. To calculate distance between the haplotypes, a haplotype evolutionary tree as shown in FIG. 6 is created. There have been reported some methods of creating the haplotype evolutionary tree such as the method shown in Document: McPeek M S & Strahs A: Assessment of linkage disequilibrium by the decay of haplotype sharing, with application to fine-scale genetic mapping, Am J Hum Genet, Vol. 65, pp. 858-875, 1999.

In the present invention, an evolutionary tree is created so that the edge of the evolutionary tree shows evolution by one mutation or one recombination. As in the evolution of haplotypes 1 to 5 of FIG. 6, when evolution cannot be expressed by one mutation or one recombination, a latent haplotype which is not actually observed is inserted to create the evolutionary tree. The haplotype 6 of FIG. 6 is an example of the latent haplotype.

For each edge of the created evolutionary tree, whether the evolution is by recombination or mutation is decided. In FIG. 6, the evolution of haplotypes 1 to 4 is considered to be by recombination. The evolution of haplotypes 1 to 2 and the evolution of haplotypes 1 to 3 are considered to be by both mutation and recombination.

The likelihood when a certain haplotype H_{S} is evolved to another haplotype H_{T} is expressed by Equation (5):

$$Pr ( {H}_{T} \vert {H}_{S} ) = Pr ( {H}_{T} \vert {H}_{S} , mut . ) Pr ( mut . \vert mut . or rec . ) + Pr ( {H}_{T} \vert {H}_{S} , rec . ) Pr ( rec . \vert mut . or rec . ) \qquad ( 5 )$$

where mut. represents mutation, and rec. represents recombination. Equation (5) shows that the likelihood when the haplotype H_{S} is evolved to the haplotype H_{T} is expressed by the sum of the likelihood when supposing that the evolution is by mutation and the likelihood when supposing that the evolution is by recombination. When a mutation rate in a certain locus j is γⱼ and a recombination rate of the kth gap in haplotype is θ, Pr(mut.|mut. or rec.)=A/(A+B) and Pr(rec.|mut. or rec.)=B/(A+B). A is as shown in Equation (6) and B is as shown in Equation (7):

$$\begin{matrix}A = {\sum }_{j} {γ}_{j} {\prod }_{i \neq j} ( 1 - {γ}_{j} ) & ( 6 ) \\ B = {\sum }_{k} {θ}_{k} {\prod }_{i \neq k} ( 1 - {θ}_{k} ) & ( 7 )\end{matrix}$$

As in the evolution of haplotypes 1 to 4 in FIG. 6, when polymorphism constructing haplotypes are different in two or more loci, the evolution is clearly by recombination and Pr(H_{T}|H_{S}, mut.)=0. In the recombination evolution, in the evolution of haplotypes 1 to 4 in FIG. 6, when recombination occurs in any gap (including both edges) on a partial haplotype GCCCTCTAT common to the right side of the haplotypes 1 and 4, the same haplotype is formed in appearance. When H_{S} and H_{T} have the same allele in appearance to the k₀th gap (called IBS (identical by state) and are different in the later part, the likelihood of recombination evolution is expressed as Equation (8):

$$Pr ( {H}_{T} \vert {H}_{S} , rec . ) = {\sum }_{k = 0}^{{k}_{0}} Pr ( {H}_{T} \vert {H}_{S} , rec . , R = k ) Pr ( R = k ) \qquad ( 8 )$$

where H_{S} is constructed by L loci and a partial haplotype constructed by parts of loci m, m+1, . . . , n of H_{S} is expressed as H_{S}^{{m:n}}. In the same manner, H_{T} is expressed by Equation (9):

$$\begin{matrix}\begin{matrix}Pr ( {H}_{T} \vert {H}_{S} , rec . , \\ R = k ) Pr ( R = k )\end{matrix} = Pr ( {H}_{T}^{1 : k} IBD to {H}_{S}^{1 : k} , \\ {H}_{T}^{( k + 1 ) : L} \vert {H}_{T}^{1 : k} IBS to {H}_{S}^{1 : k} ) \\ = Pr ( {H}_{T}^{1 : k} IBD to {H}_{S}^{1 : k} \vert {H}_{T}^{1 : k} IBS to {H}_{S}^{1 : k} ) \\ Pr ( {H}_{T}^{( k + 1 ) : L} )\end{matrix} \qquad ( 9 )$$

Here, two haplotypes being IBD (identical by descent) indicates that they have allele derived from the same ancestor. Since two haplotypes are IBS in appearance and may be actually IBD, this is expressed as IBS*.

When applying the Bayes' theorem, Equation (10) is given:

$$\begin{matrix}Pr ( {H}_{T}^{1 : k} IBD to {H}_{S}^{1 : k} \vert {H}_{T}^{1 : k} IBS to {H}_{S}^{1 : k} ) = \\ Pr ( {H}_{T}^{1 : k} IBD to {H}_{S}^{1 : k} ) / [ Pr ( {H}_{T}^{1 : k} IBD to {H}_{S}^{1 : k} ) + \\ Pr ( {H}_{T}^{1 : k} {IBS}^{*} to {H}_{S}^{1 : k} ) Pr ( {H}_{T}^{1 : k} \vert {H}_{T}^{1 : k} {IBS}^{*} to {H}_{S}^{1 : k} ) ]\end{matrix} \} \qquad ( 10 )$$

Here, Equation (11) can be supposed:

$$Pr ( {H}_{T}^{1 : k} IBD to {H}_{S}^{1 : k} ) = Pr ( {H}_{T}^{1 : k} {IBS}^{*} to {H}_{S}^{1 : k} ) = \frac{1}{2} \qquad ( 11 )$$

Since equation (12) expresses the frequency of H_{T}^{{1:k}}, the value of Equation (10) can be easily calculated:
Pr(H_{T}^{1:k}|H_{T}^{1:k}IBS* to H_{S}^{1:k}) (12)

In the present invention, the likelihood expressed by Equation (5) is newly defined as distance between haplotypes to perform clustering individuals using the distance. Distance dk between an individual having haplotypes of Hₖₐₖ, H_{kbk} and an individual having haplotypes of H_{kck}, H_{kdk} for the kth haplotype block is defined as in Equation (13):

$${d}_{k} = \begin{matrix}\frac{1}{8} [ Pr ( {H}_{{kc}_{k}} \vert {H}_{{ka}_{k}} ) + Pr ( {H}_{{ka}_{k}} \vert {H}_{{kc}_{k}} ) + \\ Pr ( {H}_{{kd}_{k}} \vert {H}_{{ka}_{k}} ) + Pr ( {H}_{{ka}_{k}} \vert {H}_{{kd}_{k}} ) + \\ Pr ( {H}_{{kc}_{k}} \vert {H}_{{kb}_{k}} ) + Pr ( {H}_{{kb}_{k}} \vert {H}_{{kc}_{k}} ) + \\ Pr ( {H}_{{kd}_{k}} \vert {H}_{{kb}_{k}} ) + Pr ( {H}_{{kb}_{k}} \vert {H}_{{kd}_{k}} )\end{matrix} \} \qquad ( 13 )$$

When the number of haplotype blocks is m, distance d between two individuals is expressed as Equation (14) by coupling distances between all haplotype blocks:

$$d = \frac{1}{m} {\sum }_{k = 1}^{m} {d}_{k} \qquad ( 14 )$$

A method of inferring a membership proportion of an individual, that is, the genetic structure inference program 15 will be described. In the present invention, information on to which subpopulation generated by the above-described clustering method each individual belongs is defined as a membership proportion of the individual.

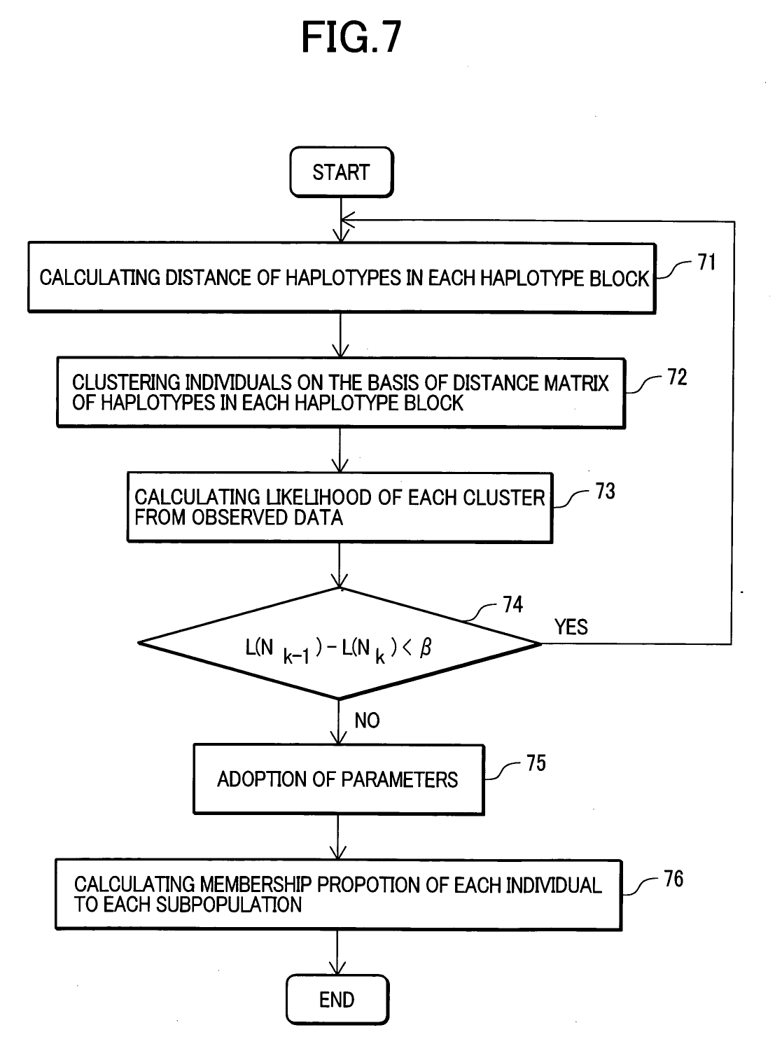

FIG. 7 is a diagram showing the genetic structure inference program 15 inferring a membership proportion of an individual.

Step 71: Distance between haplotypes in each haplotype block is decided by the method explained with reference to FIG. 6.

Step 72: Clustering on the basis of the distance between haplotypes is performed.

Step 73: From the result of step 72, a population having n individuals is divided into N subpopulations. When a certain individual i is classified into a certain subpopulation j, the membership proportion of the individual i to the subpopulation j is 100% and the membership proportion of the individual i to a subpopulation other than the subpopulation j is 0%. When the number of haplotype blocks is m, the entire likelihood can be expressed as Equation (15):

$$L ( N ) = {\prod }_{i = 1}^{n} {\sum }_{j = 1}^{N} {\prod }_{k = 1}^{m} {Pr ( D \vert G )}_{jk}^{( i )} {Q}_{j}^{( i )} \qquad ( 15 )$$

where Pr (D|G) is maximum likelihood estimation of diplotype distribution of an individual and Equation (16) shows the maximum likelihood estimation of diplotype distribution of the individual i in the kth haplotype block of the subpopulation j:
   Pr(D|G)ⱼₖ⁽ⁱ⁾ (16)

Step 74: Whether the value of L(N) is converged or not is determined. When satisfying L(Nₖ₋₁)−L(Nₖ)<β, it is converged to advance to step 75. When not satisfying it, the routine is advanced to step 71 to repeat until step 74. P is a threshold.

Equation (17) is the membership proportion of the individual i to the subpopulation j:
Qⱼ⁽ⁱ⁾ (17)

Step 75: N when the likelihood expressed by Equation (15) is maximum, is maximum likelihood estimation of the number of subpopulations. The maximum likelihood estimation is adopted as a parameter.

Step 76: The membership proportion of the individual to the subpopulation is calculated on the basis of the likelihood expressed by Equation (15). For instance, there are N_{k} subpopulations, and subpopulation N_{—}{1} is coupled to subpopulation N_{l+1} in the next link step to form N_{k−1} subpopulations. When the likelihood is not changed in this step and the likelihood is maximum, the membership proportions of all individuals classified into subpopulations N_{—}{1} and N_{l+1} to subpopulations N_{—}{1} and N_{l+1} are 50%, respectively.

As described above, the genetic structure information database 16 stores haplotype pattern and haplotype frequency information in each subpopulation and membership proportion of each individual to each subpopulation.

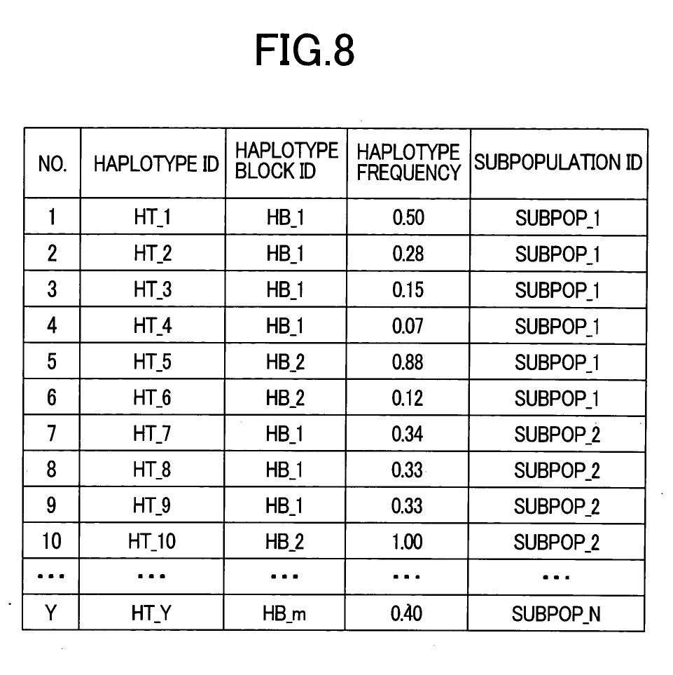

FIG. 8 is a diagram showing a storing example of haplotype pattern and haplotype frequency information in each subpopulation. For instance, there are haplotype blocks HB_1, HB_2 in subpopulations SUBPOP_1 and SUBPOP_2. Four haplotypes HT_1, HT_2, HT_3 and HT_4 exist in subpopulation SUBPOP_1. Three haplotypes HT_7, HT_8 and HT_9 exist in subpopulation SUBPOP_2.

As understood with reference to FIG. 4, for instance, four haplotypes HT_1, HT_2, HT_3 and HT_4 exist in haplotype block HB_1, and frequencies of haplotypes in the population are 0.50, 0.28, 0.15 and 0.07. Three haplotypes HT_7, HT_8 and HT 9 exist in haplotype block HB_1. Frequencies of haplotypes in the population are 0.34, 0.33 and 0.33.

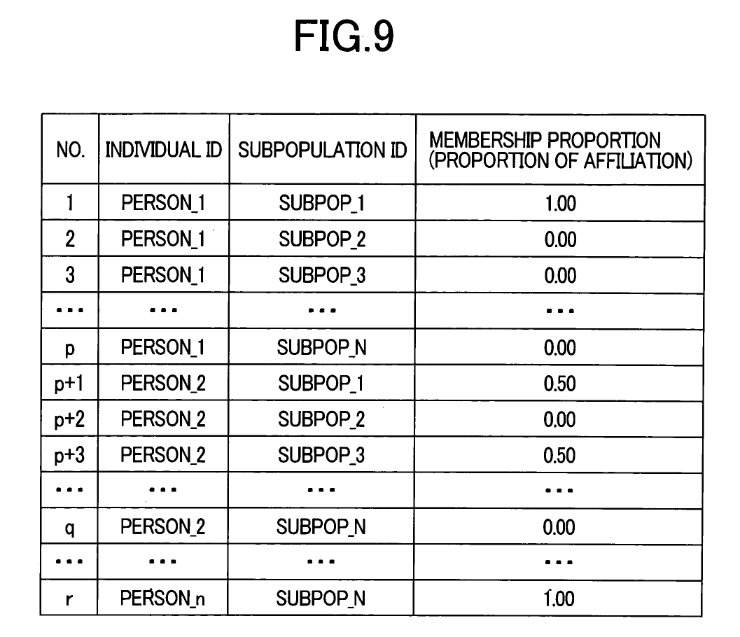

FIG. 9 is a diagram showing a storing example of membership proportion information of each individual to each subpopulation. For instance, a membership proportion of individual PERSON_1 to subpopulation SUBPOP_1 is 1.00 (which may be expressed as a percentage of 100%). A membership proportion of individual PERSON_2 to subpopulation SUBPOP_1 is 0.50 (50%). A membership proportion of individual PERSON_2 to subpopulation SUBPOP_3 is 0.50 (50%).

There will be described a procedure for analyzing association of the haplotype pattern of an individual with a trait for each haplotype block of each subpopulation on the basis of information of the clinical information database 11 and the genetic structure information database 16 by the association analysis program 17. The association analysis program 17 compares traits of a group of individuals owning a specified haplotype and a group of individuals not owning it (for instance, compares the presence or absence of disease appearing) to calculate an odds ratio of both groups, and compares the group of individuals owning a specified haplotype with the group of individuals not owning it for inferring to what degree the risk of affected disease is increased.

In the present invention, the odds ratio of disease appearing of the group of individuals owning a specified haplotype to the group of individuals not owning it is defined as a haplotype relative risk. In many cases, a 2×2 contingency table is created by the presence or absence of owning a specified haplotype and the presence or absence of disease appearing (which may be the presence or absence of a clinical event or the presence or absence of a side effect of medicine) to calculate the influence of the presence or absence of owning a specified haplotype on the presence or absence of disease appearing by a test of independence (chi-squared test or Fisher's exact test) of the 2×2 contingency table. When the traits cannot be divided into some categories, the t test or Wilcoxon test may be conducted to compare the difference in trait between the group of individuals owning a specified haplotype and the group of individuals not owning it.

Knowledge obtained by the association analysis program 17 is stored in the decision support knowledge database 18.

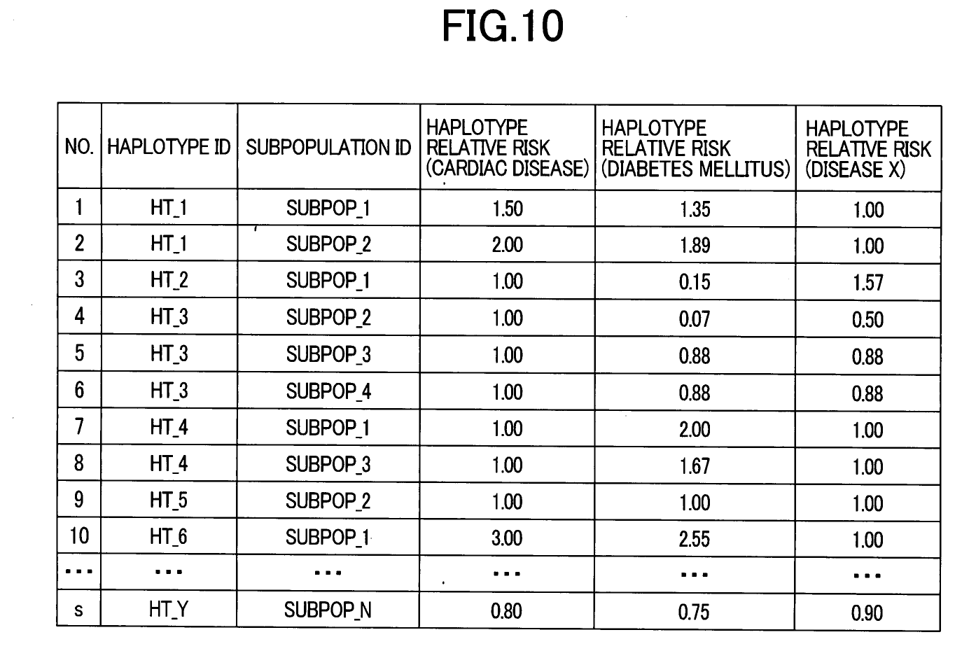

FIG. 10 is a diagram showing a description example of the decision support knowledge database 18. It shows a storing example of haplotype relative risk information in each subpopulation. The haplotype relative risk can define various clinical data such as the presence or absence of disease appearing, the presence or absence of a clinical event, normal or abnormal test result, and the presence or absence of the side effect of a medicine. Here, there is shown a storing example of haplotype relative risk information for each subpopulation to the presence or absence of appearing of cardiac disease, diabetes mellitus and disease X. In subpopulation SUBPOP_1, haplotype HT_1 has a relative risk to cardiac disease of 1.50 and relative risks to diabetes mellitus and disease X of 1.35 and 1.00. At the same time, in subpopulation SUBPOP_2, haplotype HT_1 has a relative risk to cardiac disease of 2.00 and relative risks to diabetes mellitus and disease X of 1.89 and 1.00.

The risk calculation program 19 calculates, with reference to the genetic structure information database 16 and the decision support knowledge database 18, a risk that a predetermined individual is affected by disease. Risk Rᵢ that an individual i is affected by certain disease can be expressed by Equation (18) when the number of haplotype blocks is m, the number of subpopulations existing in a population is N, and the haplotype relative risk of individual i in haplotype block k of subpopulation j is rᵢⱼₖ:

$${R}_{i} = {\prod }_{k = 1}^{m} {\sum }_{j = 1}^{N} {r}_{ijk} {Q}_{j} \qquad ( 18 )$$

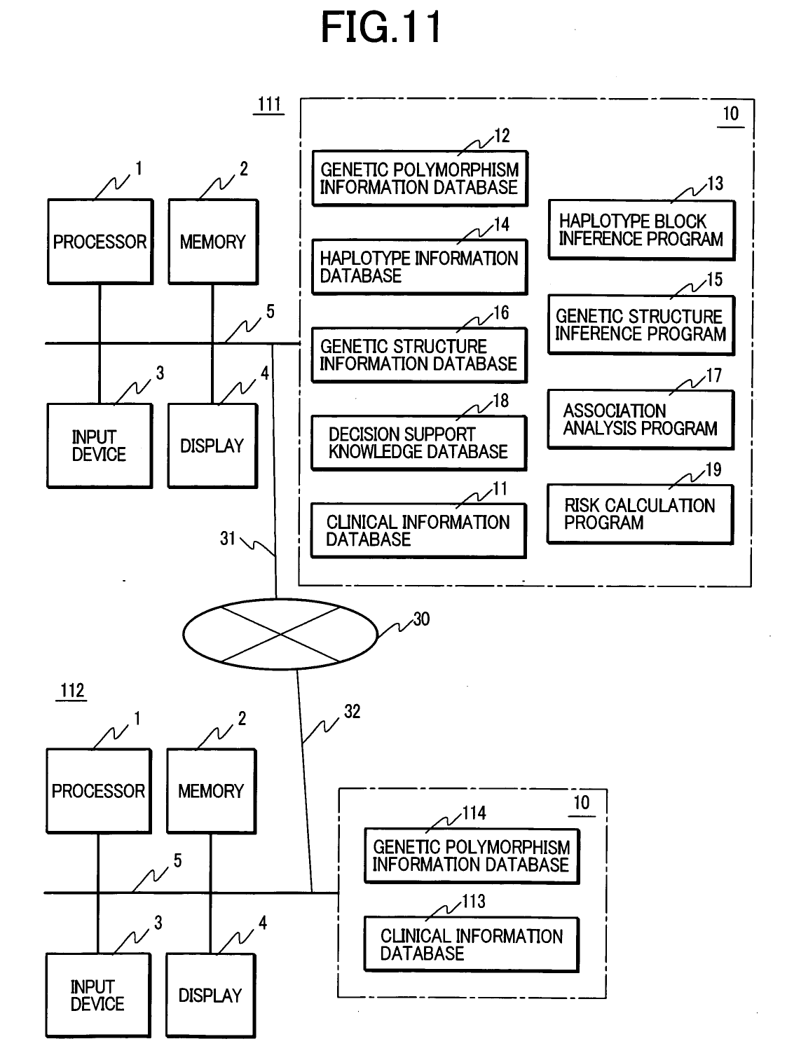

FIG. 11 is a diagram showing a system example in which an outside medical institution 112 accesses the diagnostic decision support system 111 of the present invention via connection paths 31, 32 and the Internet 30 to receive diagnostic decision support using the diagnostic decision support system 111 of the present invention. The outside medical institution 112 also has an electronic computer such as a personal computer and the system bus 5 is connected to the processor 1, the memory 2, the input device 3, the display 4, and the external memory 10. The outside medical institution 112 does not handle data of a large population unlike the present invention. The clinical information database 113 storing clinical information on a plurality of individuals (subjects) and the genetic polymorphism information database 114 storing information on polymorphism of the plurality of individuals (subjects) may be small. When the subject only receives diagnostic decision support using the diagnostic decision support system 111 of the present invention individually for diagnostic decision, the clinical information database 113 and the genetic polymorphism information database 114 may be omitted. The diagnostic decision support system 111 of the present invention is desirably more complete by collecting and providing data of subjects by the outside medical institution 112 using this to fulfill the data. When the outside medical institution 112 receives diagnostic decision support using the diagnostic decision support system 111 of the present invention, the outside medical institution 112 samples genetic data and trait data of an individual from the clinical information database 113 and the genetic polymorphism information database 114 to send them to the diagnostic decision support system 111 of the present invention. When the outside medical institution 112 does not have the clinical information database 113 and the genetic polymorphism information database 114, the information may be inputted from the input device 3 to send it to the diagnostic decision support system 111 of the present invention. The diagnostic decision support system 111 of the present invention provides calculated risk information to disease, genetic structure information and membership proportion information of an individual to each subpopulation to the outside medical institution 112 on the requiring side on the basis of the data. It is unnecessary to describe the processing flow of a computer.

1. A diagnostic decision support system comprising: a clinical information database storing clinical information on a plurality of individuals; a genetic polymorphism information database storing information on polymorphism of a population; a haplotype block inference program inferring haplotype blocks of said population and haplotype frequency in each of said haplotype blocks on the basis of information of said genetic polymorphism information database; a haplotype information database storing the haplotype pattern and said haplotype frequency in each of said inferred haplotype blocks of said population; a genetic structure inference program inferring a genetic structure existing in said population on the basis of information of said haplotype information database to divide said population into a plurality of subpopulations; a genetic structure information database storing said haplotype information for each of said divided subpopulations and membership proportion information of each of said individuals to each of said subpopulations; an association analysis program analyzing association of the haplotype with a trait of a subject on the basis of information of said clinical information database and said genetic structure information database; a database of knowledge of diagnostic decision support storing information obtained by said association analysis program; and a risk calculation program calculating, on the basis of information of said database of knowledge of diagnostic decision support, a risk that a predetermined individual is affected by disease.

2. The diagnostic decision support system according to claim 1, wherein said genetic structure inference program performs a process for performing clustering on the basis of a distance defined between haplotypes existing in each of said haplotype blocks, a process for obtaining said haplotype pattern and said haplotype frequency for each of said subpopulations obtained by said clustering, a process for determining a suitable number of said subpopulations, and a process for obtaining a membership proportion of each of said individuals to said obtained subpopulation.

3. The diagnostic decision support system according to claim 2, wherein said distance is defined by the likelihood of recombination and mutation between haplotypes.

4. A method of diagnostic decision support comprising the steps of: inferring haplotype blocks and haplotype frequency in each of the haplotype blocks on the basis of information of a genetic polymorphism information database storing information on polymorphism; storing a haplotype pattern and the haplotype frequency in each of said inferred haplotype blocks in a haplotype information database; inferring a genetic structure existing in a population on the basis of information of said haplotype information database to infer a genetic structure dividing said population into a plurality of subpopulations; storing said haplotype information for each of said divided subpopulations and membership proportion information of each of said individuals to each of said subpopulations in a genetic structure information database; analyzing association of a haplotype with a trait on the basis of information of the clinical information database storing clinical information on a plurality of individuals and said genetic structure information database; storing information obtained by said association analyzing step in a database of knowledge of diagnostic decision support; and calculating, on the basis of information of said database of knowledge of diagnostic decision support, a risk that a predetermined individual is affected by disease.

5. The method of diagnostic decision support according to claim 4, wherein said step of inferring a genetic structure performs a process for performing clustering on the basis of a distance defined between haplotypes existing in each of said haplotype blocks, a process for obtaining said haplotype pattern and said haplotype frequency for each of said subpopulations obtained by said clustering, a process for determining a suitable number of said subpopulations, and a process for obtaining a membership proportion of each of said individuals to said obtained subpopulation.

6. The method of diagnostic decision support according to claim 5, wherein said distance is defined by the likelihood of recombination and mutation between haplotypes.

7. A diagnostic decision support service which can be received by being connected to a diagnostic decision support system comprising a clinical information database storing clinical information on a plurality of individuals; a genetic polymorphism information database storing information on polymorphism; a haplotype block inference program inferring haplotype blocks and haplotype frequency in each of said haplotype blocks on the basis of information of said genetic polymorphism information database; a haplotype information database storing a haplotype pattern and said haplotype frequency in each of said inferred haplotype blocks; a genetic structure inference program inferring a genetic structure existing in a population on the basis of information of said haplotype information database to divide said population into a plurality of subpopulations; a genetic structure information database storing said haplotype information for each of said divided subpopulations and membership proportion information of each of said individuals to each of said subpopulations; an association analysis program analyzing association of the haplotype with a trait on the basis of information of said clinical information database and said genetic structure information database; a database of knowledge of diagnostic decision support storing information obtained by said association analysis program; and a risk calculation program calculating, on the basis of information of said database of knowledge of diagnostic decision support, a risk that a predetermined individual is affected by disease, wherein a person receiving the diagnostic decision support service transmits, to the diagnostic decision support system, genotype data and trait data of said predetermined individual received from the individual as a subject, and the diagnostic decision support system calculates information on a genetic structure existing in said population, a membership proportion of said predetermined individual to each of said subpopulations, and a risk that said predetermined individual is affected by disease for providing them to said person receiving the diagnostic decision support service.
